(19) United States
(12) Patent Application Publication
(10) Pub. No.: US 2010/0065068 A1
(43) Pub. Date: Mar. 18, 2010

(54) DRAPE FOR MEDICAL EQUIPMENT, DRAPE FOR ENDOSCOPE, AND MEDICAL APPARATUS

(76) Inventors: Masanori HAMAZAKI, Hachioji-shi (JP); Fumiyuki ONODA, Tama-shi (JP); Katsuyuki SAITO, Sagamihara-shi (JP); Seiji YAMAGUCHI, Hachioji-shi (JP); Kiyoshi MIYAKE, Niiza-shi (JP)

Correspondence Address: OSTROLENK FABER GERB & SOFFEN, 1180 AVENUE OF THE AMERICAS, NEW YORK, NY, 100368403, US

(21) Appl. No.: 12/620,078

(22) Filed: Nov. 17, 2009

Related U.S. Application Data
(63) Continuation of application No. PCT/JP2007/062723, filed on Jun. 25, 2007.

(51) Int. Cl. A61B 19/10

(52) U.S. Cl. 128/849

(57) ABSTRACT

A drape for a medical equipment includes a covering portion configured to cover a surface of the medical equipment, a fixing portion which relatively fixes the covering portion to the medical equipment, a coupling portion which couples the fixing portion with the covering portion, and holding means which is provided to the covering portion and configured to liquid-tightly hold the covered inside when the medical equipment is covered with the covering portion.

### CROSS-REFERENCE TO RELATED APPLICATIONS

This is a Continuation Application of PCT Application No. PCT/JP2007/062723, filed Jun. 25, 2007, which was published under PCT Article 21(2) in Japanese.

### BACKGROUND OF THE INVENTION

1. Field of the Invention

The present invention relates to a drape for a medical equipment, a drape for an endoscope, and a medical apparatus used for various medical equipment.

2. Description of the Related Art

For example, when discarding a medical apparatus such as a disposable endoscope or treatment tool having blood, bacteria and so on attached thereto after a treatment, a bag is prepared, and the used medical equipment is put into the bag to be discarded.

Further, for example, JP-H06-245897 A1 (KOKAI) discloses that an endoscope is covered with a cover at the time of a treatment (use). The cover is reversed to cover an endoscope when using the endoscope, and is restored to its original state from the reversed state and disconnected from the endoscope to be discarded after use of the endoscope.

### BRIEF SUMMARY OF THE INVENTION

A drape for a medical equipment according this invention includes: a covering portion configured to cover a surface of the medical equipment; a fixing portion which relatively fixes the covering portion to the medical equipment; a coupling portion which couples the fixing portion with the covering portion; and

holding means which is provided to the covering portion and configured to liquid-tightly hold the covered inside when the medical equipment is covered with the covering portion.

A drape for an endoscope according this invention includes: a covering portion configured to change from a state where at least a part of an operation portion of the endoscope having an insertion portion and the operation portion provided at a proximal end portion of the insertion portion is covered to a state where the entire insertion portion is covered; a fixing portion which relatively fixes the covering portion to the endoscope; a coupling portion which couples the fixing portion to the covering portion; and holding means which is provided to the covering portion and configured to liquid-tightly cover the entire insertion portion in a state where the entire insertion portion is covered with the covering portion.

A medical apparatus according this invention includes: a control apparatus; an endoscope having an insertion portion and an operation portion which is provided at a proximal end portion of the insertion portion and has an attachment/detachment portion detachably connected with the control apparatus; a covering portion configured to change from a state where at least a part of the operation portion is covered to a state where the entire insertion portion is liquid-tightly covered; a fixing portion which relatively fixes the covering portion to the attachment/detachment portion; and holding means for enabling the covering portion to cover a surface of the control apparatus.

### BRIEF DESCRIPTION OF THE SEVERAL VIEWS OF THE DRAWING

FIG. 1A is a schematic view showing an endoscope system according to a first embodiment of the present invention;

FIG. 1B is a schematic plan view showing an end face of a distal end hard portion of an insertion portion of an endoscope in the endoscope system according to the first embodiment denoted by reference character 1B in FIG. 1A;

FIG. 2A is a schematic view showing a drape that covers a video processor, a light source apparatus, and a monitor in the endoscope system according to the first embodiment;

FIG. 2B is a schematic partial cross-sectional view showing a state where a concave groove on an outer peripheral surface of a universal cable of the endoscope is engaged with a coupling portion (an opening) of the drape in the endoscope system according to the first embodiment;

FIG. 3A is a schematic view showing an endoscope in an endoscope system according to a second embodiment of the present invention;

FIG. 3B is a schematic partial cross-sectional view showing a state where a concave groove on an outer peripheral surface of a light guide cable connected with the endoscope is engaged with a coupling portion (an opening) of a drape in the endoscope system according to the second embodiment;

FIG. 4A is a schematic partial cross-sectional view showing a state where a drape is arranged and accordion-folded in a connector of a universal cable of an endoscope in a contracted state in an endoscope system according to a third embodiment of the present invention; and

FIG. 4B is a schematic partial cross-sectional view showing both an extended state where the drape arranged in the connector of the universal cable of the endoscope is extended to a distal end portion of an insertion portion of the endoscope and a state where an end of the drape is closed in the endoscope system according to the third embodiment.

### DETAILED DESCRIPTION OF THE INVENTION

The best modes for carrying out the present invention will now be described hereinafter with reference to the drawings.

### First Embodiment

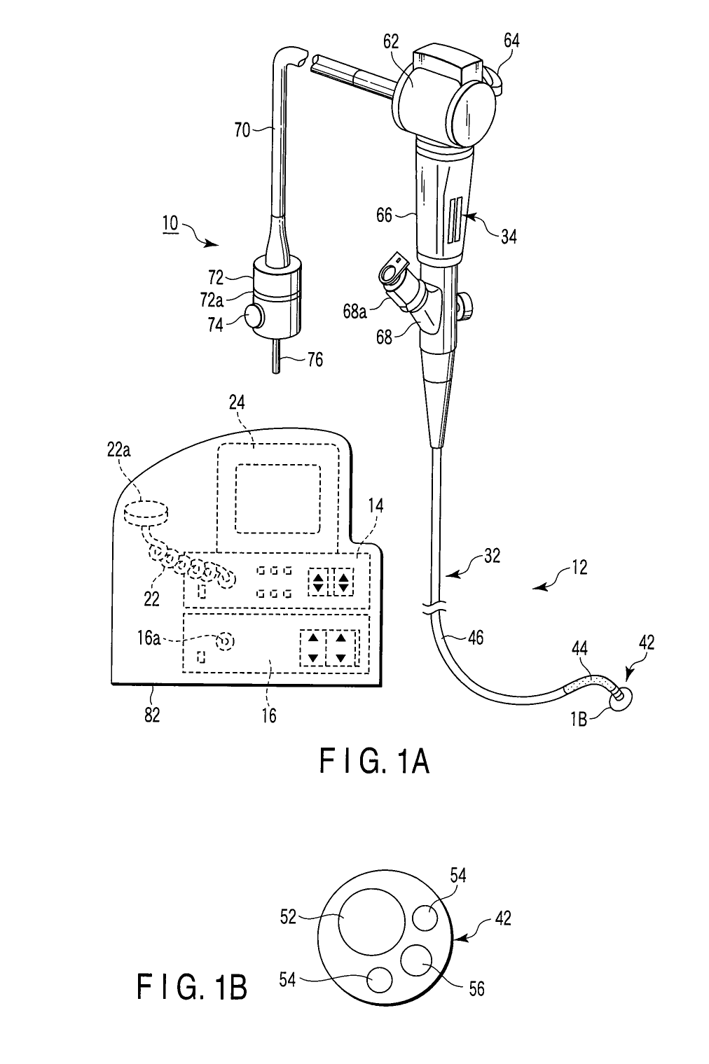

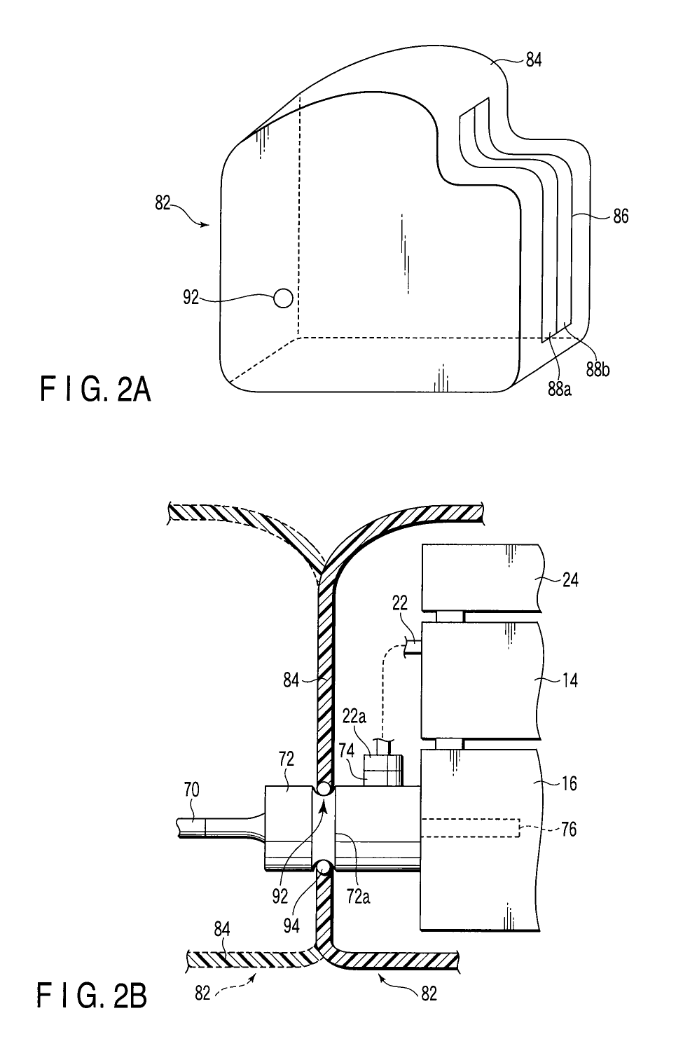

A first embodiment will be first described with reference to FIGS. 1A to 2B.

As shown in FIGS. 1A and 1B, an endoscope system 10 as a medical apparatus according to this embodiment includes an endoscope 12 as a medical apparatus, and a video processor 14 and a light source apparatus 16 which are medical equipment control apparatuses that can be attached to/detached from the endoscope 12. A scope cable 22 and a monitor 24 are connected with the video processor 14.

The endoscope 12 according to this embodiment is used as a disposable type, but its main configuration is the same as a configuration which is cleaned, sterilized, and disinfected for reutilization. That is, as shown in FIG. 1A, the endoscope 12 includes an insertion portion (an application unit which is directly applied to a living tissue) 32 which is inserted into an observation region, and an operation portion (a non-application unit which is not applied to a living tissue) 34 provided at a proximal end of the insertion portion 32.

The insertion portion 32 sequentially includes a distal end hard portion 42, a bending portion 44 which is bent via an operation from the operation portion 34, and a flexible portion 46 which has flexibility from its distal end portion side. The insertion portion 32 includes an illumination optical system having, e.g., a forceps channel 52 (see FIG. 1B), an illumination fiber cable for guiding illumination light, an illumination lens 54, and other parts, and an observation optical system having, e.g., an object lens 56, a CCD, a signal line through which a signal from the CCD is transmitted, and other parts therein. As shown in FIG. 1B, a distal end opening of the forceps channel 52, the illumination lens 54 at an end of the illumination fiber cable in the illumination optical system, and the object lens 56 at the end of the observation optical system are arranged on a distal end face of the distal end hard portion 42.

The operation portion 34 includes an operation portion main body 62, an angle lever 64 which operates the bending portion 44 in the insertion portion 32 to bend, a grip 66 held by an operator, a forceps mouth ring 68, and a universal cable 70 extended from the operation portion main body 62. The angle lever 64 is provided to the operation portion main body 62. The operation portion main body 62 is arranged at an upper end of the grip 66 in FIG. 1A, and the forceps mouth ring 68 is arranged at a lower end of the grip 66. The forceps mouth ring 68 has a forceps insertion opening communicating with the forceps channel 52 of the insertion portion 32. The forceps insertion opening of the forceps mouth ring 68 is closed by a forceps tap 68a when a forceps is not inserted into the insertion portion 32.

When operating the endoscope 12, an operator (an observer) holds the grip 66 of the operation portion 34 and bends the bending portion 44 of the insertion portion 32 by using the angle lever 64 of the operation portion 34 to insert the insertion portion 32 into an observation region. Further, the observation region is illuminated with illumination light emitted from the illumination lens 54 of the distal end hard portion 42. The illuminated observation region is captured by the object lens 56 in the observation optical system and displayed in the monitor 24 via the scope cable 22 and the video processor 14 through the observation optical system in the endoscope 12.

The universal cable 70 is covered with a resin material such as polyurethane. A connector (an attachment/detachment portion) 72 formed of a hard resin material is disposed at an end portion of the universal cable 70 which is a far end with respect to the operation main portion body 62. The connector 72 includes an electrical first connection unit 74 which can be attached to/detached from the connector 22a of the scope cable 22 of the video processor 14 and an optical second connection unit 76 which can be attached to/detached from the light source apparatus 16. It is to be noted that a concave groove 72a having a substantially annular shape is formed on an outer peripheral surface of the connector 72. The concave groove 72a is formed on a side close to the operation portion 34 rather than the first and second connection units 74 and 76.

As shown in FIG. 1A, the video processor 14, the light source apparatus 16, and the monitor 24 are covered with a drape 82 to avoid adhesion of bacteria, etc. As shown in FIG. 2A, the drape 82 includes a covering portion 84, an opening portion 86, and, e.g., hook-and-loop fasteners 88a and 88b as opening/closing portions for opening/closing the opening portion 86 (a holding unit or holding means that can liquid-tightly hold the inside of the covering portion 84). The covering portion 84 covers the video processor 14, the light source apparatus 16, and the monitor 24. The video processor 14, the light source apparatus 16, and the monitor 24 are put in/taken out through the opening portion 86. For example, the hook-and-loop fasteners 88a and 88b are arranged in the opening portion 86. Therefore, the drape 82 is sealed when the hook-and-loop fasteners 88a and 88b are fastened, and the drape 82 is opened when the hook-and-loop fasteners 88a and 88b are released.

Moreover, a coupling portion (an opening) 92 in which the connector 72 of the universal cable 70 in the endoscope 12 is arranged in a closely attached manner is formed in the drape 82. As shown in FIG. 2B, in the coupling portion 92, an O-ring 94 which can elastically deform to be expanded and contracted in a radial direction is arranged as, e.g., a fixing portion which realizes detachable fixation to the connector 72 of the universal cable 70 of the endoscope 12. Therefore, the coupling portion 92 to which the O-ring 94 is arranged is attached to the concave groove 72a of the connector 72 of the universal cable 70 of the endoscope 12.

A function of the endoscope system 10 according to this embodiment will now be described.

As shown in FIG. 1A, the video processor 14, the light source apparatus 16, and the monitor 24 are covered with the drape 82, and then the hook-and-loop fasteners 88a and 88b depicted in FIG. 2A are engaged with each other. Further, the first connection unit 74 of the connector 72 of the universal cable 70 is connected with the connector 22a of the scope cable 22, and the second connection unit 76 is connected with a connector receiving portion 16a of the light source apparatus 16.

At this time, as shown in FIG. 2B, the O-ring 94 of the coupling portion 92 in the drape 82 is arranged in the concave groove 72a on the connector 72 of the universal cable 70. Therefore, the coupling portion 92 of the drape 82 keeps the inside of the drape 82 away from the outside of the same.

In this state, a treatment is carried out like a regular medical endoscope 12. Thereafter, the endoscope 12 according to this embodiment is discarded since it is a disposable type. A method (a function) of discarding the endoscope 12 will now be described hereinafter.

First, the connection achieved between the first connection unit 74 of the connector 72 of the universal cable 70 and the connector 22a of the scope cable 22 is released, and the connection between the second connection unit 76 and the connector receiving portion 16a of the light source apparatus 16 is released. In this state, the engagement between the hook-and-loop fasteners 88a and 88b is also released. Furthermore, the video processor 14, the light source apparatus 16, and the monitor 24 are exposed from the drape 82 while reversing the drape 82. At this time, the endoscope 12 is covered with the reversed drape 82 while keeping the connection between the connector 72 of the endoscope 12 and the drape 82. In this state, the hook-and-loop fasteners 88a and 88b are again engaged. Therefore, the endoscope 12 is hermetically housed. The endoscope 12 arranged in the drape 82 in this manner is discarded as it is.

It is to be noted that evacuating air from, e.g., a space between the hook-and-loop fasteners 88a and 88b as required is also referable.

As explained above, according to this embodiment, the following effects can be obtained.

When the drape 82 that prevents blood, bacteria and so on from being attached to the video processor 14, the light source apparatus 16, and the monitor 24 during a treatment of the endoscope 12 is reversed after the treatment of the endoscope 12, a surface of the drape 82 which is provided outside during the treatment using the endoscope 12 and may possibly have blood, bacteria and so on attached thereto can be set inside to wrap the endoscope 12. At this time, the drape 82 can be reversed while keeping the connection between the endoscope 12 and the drape 82. Therefore, the inside of the drape 82 can be hermetically sealed like the state before the reversal, and blood, bacteria and so on can be prevented from spreading as much as possible when the endoscope 12 is wrapped. Additionally, since the endoscope 12 can be discarded in this state, a bag into which the endoscope 12 is put does not have to be additionally prepared, thereby reducing a cost for an operation.

It is to be noted that the bending portion 44 of the insertion portion 32 of the disposable endoscope 12 can bend in two directions in the description of this embodiment. Further, configuring the bending portion 44 to bend in four directions is also possible.

Further, the case where the hook-and-loop fasteners 88a and 88b are used to seal the drape 82 has been described in this embodiment. Besides, it is also possible to use, e.g., slide fasteners that can close a bag or the like by moving sliders.

Furthermore, although the disposable endoscope 12 has been described in this embodiment, the present invention can be also applied to treatment tools which are utilized solely or together with the endoscope 12 (e.g., catheter, a trocar, a drain tube, and other tools each having an insertion portion which is inserted into a living tissue and a proximal portion provided at a proximal end of the insertion portion).

Moreover, the drape 82 may be directly disposed to the universal cable 70 through the fixing portion without providing the coupling portion 92. Additionally, the drape 82 may be provided at any part of the endoscope 12 except the insertion portion 32 of the endoscope 12 which is applied to a living body.

### Second Embodiment

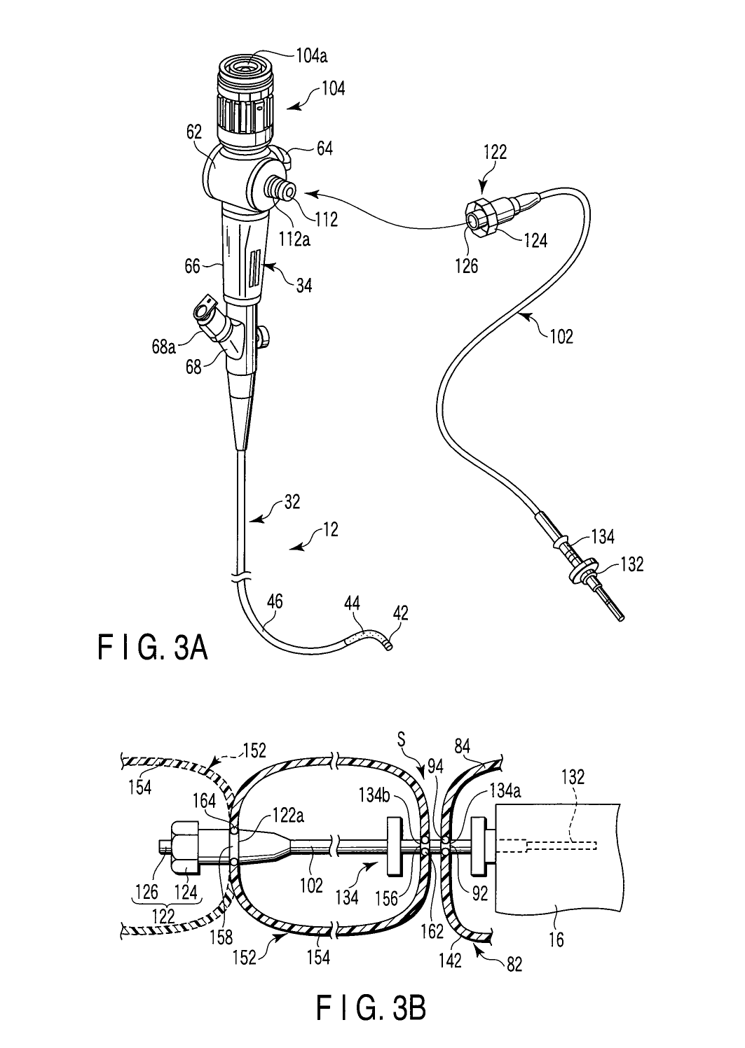

A second embodiment will now be described with reference to FIGS. 3A and 3B. This embodiment is a modification of the fist embodiment, and like reference numerals denote members equal to those described in the first embodiment to omit a detailed explanation thereof.

As shown in FIG. 3A, an endoscope system 10 as a medical apparatus according to this embodiment includes an endoscope 12 and a light source apparatus 16. That is, the video processor 14, the scope cable 22, and the monitor 24 in FIG. 1A described in the first embodiment are removed from the endoscope system 10.

As shown in FIG. 3A, in this embodiment, the universal cable 70 is removed from the operation portion main body 62 of the operation portion 34 in the endoscope 12, and a light guide cable 102 and an eyepiece unit 104 are arranged in the operation portion main body 62 instead. That is, this embodiment includes, e.g., an object lens 56, an image guide fiber, and the eyepiece unit 104 as an observation optical system. A proximal end of the image guide fiber internally provided from a distal end hard portion 42 of an insertion portion 32 to the operation portion main body 62 is arranged in the eyepiece unit 104. Further, the eyepiece unit 104 includes an eyepiece lens 104a at the proximal end of the image guide fiber.

On the other hand, in an illumination optical system, an end portion of a light guide fiber is provided at the operation portion main body 62 since the universal cable 70 is removed. An endoscope-side light source mouth ring 112 to which the end portion of the light guide fiber is fixed is arranged on the operation portion main body 62. The endoscope-side light source mouth ring 112 includes a screw portion 112a on which a later-explained light source socket 122 of the light guide cable 102 is threadably mounted in order to lead light from the outside of the endoscope 12. Further, the light source apparatus 16 having, e.g., high luminance is connected to the endoscope-side light source mouth ring 112 of the operation portion main body 62 through the light guide cable 102. That is, the light guide cable 102 can be attached to/detached from the operation portion main body 62.

The light source socket 122 which is connected with the endoscope-side light source mouth ring 112 of the operation portion main body 62 is provided at an end of the light guide cable 102. The light source socket 122 includes a cap 124 having a screw portion which is screwed to the screw portion 112a provided to the endoscope-side light source mouth ring 112 and a condenser lens 126 which condenses illumination light from the light source apparatus 16.

An external light source connector 132 connected with the light source apparatus 16 and a connector holding portion 134 of the connector 132 are provided at the other end of the light guide cable 102.

Therefore, when the external light source connector 132 is connected with a connector receiving portion 16a of the light source apparatus 16, the light source apparatus 16 is optically connected with the endoscope 12 including the light guide cable 102. That is, when the light source apparatus 16 is connected with the endoscope-side light source mouth ring 112 of the operation portion main body 62 through the light guide cable 102, the endoscope-side light source mouth ring 112 is irradiated with illumination light from the light source apparatus 16 through the condenser lens 126 of the light source socket 122 of the light guide cable 102. Then, the illumination light exits from an illumination lens 54 at the end of the insertion portion 32 of the endoscope 12 through a grip 66 and the light guide fiber in the insertion portion 32. It is to be noted that two concave grooves 134a and 134b are substantially annularly formed on an outer peripheral surface of the connector holding portion 134.

Further, in this embodiment, a drape (a light source apparatus drape) 142 is arranged on the light source apparatus 16. The drape 142 includes a covering portion 84 like the drape 82 described in the first embodiment, and a coupling portion (an opening) 92 is formed in the covering portion 84. As shown in FIG. 3B, for example, an O-ring 94 is arranged in the coupling portion 92. Therefore, the coupling portion 92 having the O-ring 94 arranged therein is attached to the concave groove 134a on the connector holding portion 134 of the light guide cable 102.

Furthermore, a drape (a light guide drape) 152 which prevents blood, bacteria and so on from being attached to the light guide cable 102 as much as possible is arranged on the outer side of the light guide cable 102. The drape 152 includes a cylindrical covering portion 154, a first coupling portion (an opening) 156 provided at one end of the covering portion 154, and a second coupling portion 158 provided at the other end of the coupling portion 154.

The coupling portion (an opening) 156 in which the concave groove 134b of the connector holding portion 134 at one end of the light guide cable 102 is very closely arranged is formed at one end of the drape 152. As shown in FIG. 3B, an O-ring 162 which can elastically deform to be expanded and contracted in, e.g., a radial direction is arranged in the coupling portion 156 as a fixing portion that realizes detachable fixation to the connector holding portion 134 of the light guide cable 102 in the endoscope 12. Therefore, the coupling portion 156 having the O-ring 162 arranged therein is disposed to the connector holding portion 134 of the light guide cable 102.

The coupling portion (an opening) 158 in which the light source socket 122 at the other end of the light guide cable 102 is very closely arranged is formed at the other end of the drape 152. As shown in FIG. 3B, an O-ring 164 which can elastically deform to be expanded and contracted in, e.g., the radial direction is arranged in the coupling portion 158 as a fixing portion which realizes detachable fixation to the light source socket 122 of the light guide cable 102 in the endoscope 12. Therefore, the coupling portion 158 in which the O-ring 164 is arranged is disposed to the concave groove 122a of the light source socket 122 of the light guide cable 102.

A function of the endoscope system 10 according to this embodiment will now be described.

The outer periphery of the light guide cable 102 is covered with the drape 152. At this time, the O-ring 164 is attached to the concave groove 122a on the light source socket 122 side, and the O-ring 162 is attached to the concave groove 134b close to the light source socket 122 in the concave grooves 134a and 134b on the external light source connector 132 side. The light source apparatus 16 is covered with the drape 142.

The cap 124 of the light source socket 122 of the light guide cable 102 in this state is connected with the screw portion 112a of the endoscope-side light source mouth ring 112 of the operation portion main body 62 in the endoscope 12. Furthermore, the external light source connector 132 of the light guide cable 102 is attached to the connector receiving portion 16a through the coupling portion 92. Moreover, the O-ring 94 is attached to the concave groove 134a close to the external light source connector 132 in the concave grooves 134a and 134b on the external light source connector 132 side of the light guide cable 102.

An appropriate treatment is carried out by using the endoscope 12 in this state. After the end of the treatment, the endoscope 12 is discarded. At this time, in the drape 152 that covers the light guide cable 102, the O-ring 162 is detached from the concave groove 134b on the external light source connector 132 side. Moreover, the end portion of the drape 152 at which the O-ring 162 is arranged is moved closer to the operation portion main body 62 of the endoscope 12 while reversing the drape 152.

Additionally, the operation portion main body 62, the grip 66, and the insertion portion 32 of the endoscope 12 are sequentially covered with the end portion detached from the concave groove 134b while maintaining the attachment of the O-ring 164 at the end portion of the drape 152 with respect to the concave groove 122a of the light source socket 122 of the light guide cable 102. Further, in the drape 152, the end portion on the distal end side of the insertion portion 32 of the endoscope 12 is first hermetically closed. Then, the attachment of the cap 124 of the light source socket 122 is released with respect to the screw portion 112a of the endoscope-side light source mouth ring 112 of the light guide cable 102. Furthermore, the O-ring 164 at the end portion of the drape 152 is detached from the concave groove 122a of the light source socket 122 of the light guide cable 102. Moreover, in the drape 152, the end portion on the operation portion main body 62 side of the endoscope 12 is hermetically closed. In this state, the endoscope 12 is discarded.

On the other hand, the light guide cable 102 removed from the operation portion main body 62 is cleaned, sterilized, and disinfected for reutilization.

As explained above, according to this embodiment, the following effect can be obtained.

Since the light guide cable 102 is configured to be removable for reutilization, the light guide cable 102 can be reutilized by, e.g., cleaning, sterilizing, and disinfecting the light guide cable 102. Therefore, a cost of the endoscope 12 can be reduced. Additionally, since the endoscope 12 can be covered with the drape 152 that covers the light guide cable 102 and the endoscope 12 can be discarded, a moving distance of the drape 152 can be reduced to be shorter than that of the drape 142 arranged in, e.g., the light source apparatus 16. Accordingly, the endoscope 12 can be covered with the drape 152 to be easily discarded while preventing, e.g., blood, bacteria and so on from spreading from the endoscope 12 and other items.

It is to be noted that the case where the eyepiece unit 104 is provided to the operation portion main body 62 has been descried in this embodiment, but enabling arranging a non-illustrated camera to the eyepiece unit 104 is also preferable. Therefore, although not shown, an endoscopic image can be displayed in the monitor. In this case, as described in the first embodiment in conjunction with FIG. 1A, arranging the video processor 14, the light source apparatus 16, and the monitor 24 and arranging the drape 82 with respect to these members is preferable.

Further, although FIG. 3B shows a according this invention includes: state where the concave grooves 134a and 134b of the light guide cable 102 are separated from each other and the drapes 142 and 152 have a gap S that prevents these drapes 142 and 152 from interfering with each other, a state where the drapes 142 and 152 themselves interfere with each other in the concave grooves 134a and 134b is also possible. In this case, the gap S between the concave grooves 134a and 134b can be removed by producing a portion where the drapes 142 and 152 overlap. At this time, arranging the drape 152 on the outer side of the drape 142 is preferable.

Furthermore, a description has been given as to the case where the light guide cable 102 is subjected to reutilization in this embodiment. When discarding the endoscope together with the light guide cable 102, the endoscope 12 as well as the drape 152 can be tied with the light guide cable 102 after use of the endoscope 12, thereby maintaining the endoscope 12 to be discarded in a desired state. Alternatively, when the light guide cable 102 substitutes for the universal cable 70 described in the first embodiment, the endoscope 12 including the light guide cable 102 can be discarded as explained in the first embodiment.

Further, the case where the drapes 142 and the 152 have the gap S therebetween has been described in this embodiment, although fixing edges of the coupling portions 92 and 156 by using, e.g., an adhesive or providing slide fasteners or the like to enable attachment/detachment of these coupling portions is also possible. That is, fixing the drapes 142 and 152 or enabling attachment/detachment of the drapes 142 and 152 is also possible. In this case, the O-rings 94 and 162 do not have to be provided. In this case, not only the drape 152 but also the covering portion 154 of the drape 152 can be reversed to cover the endoscope 12, as described in the first embodiment.

Furthermore, although the description has been given as to the case where the O-rings 162 and 164 are used as fixing portions which achieve detachable fixation to the light source socket 122 or the connector holding portion 134 of the light guide cable 102 in this embodiment, thin wires such as cords that realize detachable fixation to the light source socket 122 or the connector holding portion 134 can be also used. Moreover, likewise, a part of the covering portion 154 itself of the drape 152 can be used to tie the light source socket 122 or the connector holding portion 134 of the light guide cable 102 in place of the O-rings 162 and 164.

### Third Embodiment

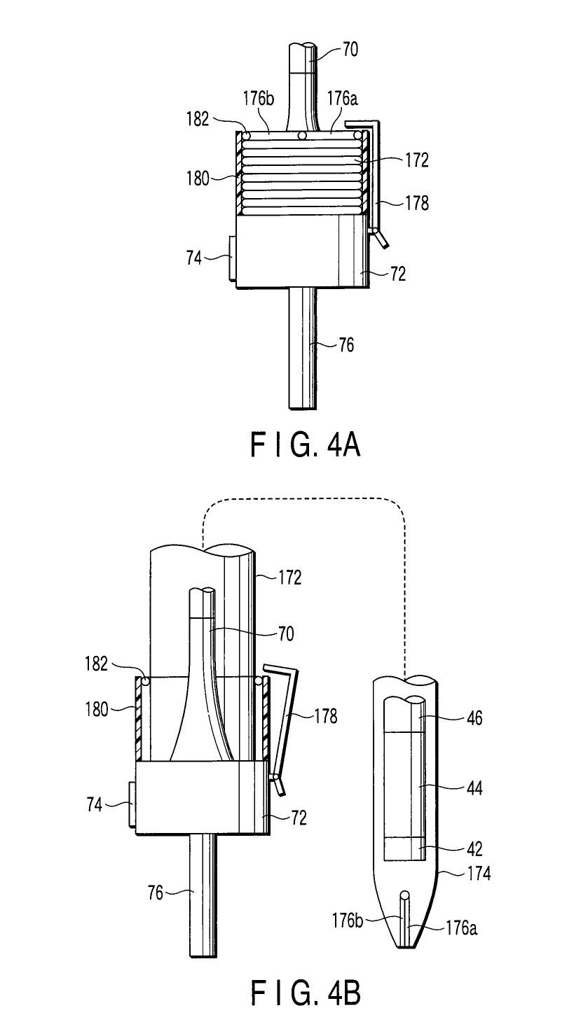

A third embodiment will now be described with reference to FIGS. 4A and 4B. This embodiment is a modification of the first embodiment, and like reference numerals denote members equal to those described in the first embodiment, thereby omitting a detailed description thereof.

As shown in FIG. 4A, a drape 172 is arranged at an extended end portion of the universal cable 70. The drape 172 includes a cylindrical accordion-folded covering portion (an elastic portion) 174 and a pair of metal fittings 176a and 176b. The drape 172 is formed with a length longer than a total length of an operation portion 34 having the universal cable 70 and an insertion portion 32 of the endoscope 12 when the covering portion 174 is extended. A proximal end of the covering portion 174 of the drape 172 is fixed to a connector 72 of the universal cable 70 by a portion denoted by reference numeral 174a.

The pair of hinged semicircular metal fittings 176a and 176b are arranged at an end of the covering portion 174 of the drape 172. Moreover, of these metal fittings 176a and 176b, one metal fitting 176a is engaged by a release lever 178 arranged on the connector 72, thereby folding the covering portion 174.

It is to be noted that the accordion drape 172 is accommodated in, e.g., a cylindrical member 180 provided to the connector 72 in order to prevent, e.g., blood, bacteria and so on from being attached as much as possible during an operation. Additionally, to avoid blood, bacteria and so on from being attached to an outer side of the drape 172, outer peripheral surfaces of the metal fittings 176a and 176b are closely arranged on an inner peripheral surface of the cylindrical member 180 through, e.g., an O-ring 182 as a sealing member that closes a space between the inner peripheral surface of the cylindrical member 180 and the metal fittings 176a and 176b, and an end portion of the drape 172 to which the metal fittings 176a and 176b are fixed is opened.

When the release lever 178 is operated, the engagement between the metal fitting 176a and the release lever 178 is released, and the drape 172 can be extended to the distal end side of the insertion portion 32 through the operation portion 34 having the universal cable 70 and the insertion portion 32 of the endoscope 12.

Therefore, when the release lever 178 is operated to unfold the accordion drape 172 and the drape 172 is extended toward the distal end portion of the insertion portion 32 of the endoscope 12, the outer peripheries of the operation portion 34 having the universal cable 70 and the insertion portion 32 of the endoscope 12 can be covered.

Further, after the entire endoscope 12 is covered with the drape 172, the end of the drape 172 is closed and sealed by closing the metal fittings 176a and 176b.

As explained above, according to this embodiment, the following effect can be obtained.

In a state where the folded drape 172 is fixed at the extended end portion of the universal cable 70, when the drape 172 is opened by operating the release lever 178 engaged with the metal fittings 176a and 176b of the drape 172, the drape 172 can readily cover the entire endoscope 2. At this time, when the metal fittings 176a and 176b are appressed against the cylindrical member 180 through the O-ring 182, blood, bacteria and so on can enter the drape 172 during an operation, but can be prevented from being attached to the outer peripheral surface of the drape 172 as much as possible. Therefore, when the drape 172 is extended to cover the endoscope 12, blood, bacteria and so on can be prevented from being attached to the outer peripheral surface of the drape 172.

Although the several embodiments have been specifically explained with reference to the accompanying drawings, the present invention is not restricted to the foregoing embodiments, and includes all embodiments carried out without departing from the scope of the invention.

What is claimed is:

1. A drape for a medical equipment comprising:
a covering portion configured to cover a surface of the medical equipment;
a fixing portion which relatively fixes the covering portion to the medical equipment;
a coupling portion which couples the fixing portion with the covering portion; and
holding means which is provided to the covering portion and configured to liquid-tightly hold the covered inside when the medical equipment is covered with the covering portion.

2. The drape for the medical equipment according to claim 1, wherein the fixing portion includes an attachment portion which is attached to a non-applying portion that is different from an applying portion directly applied to a living tissue and is not applied to a living tissue in the medical equipment.

3. The drape for the medical equipment according to claim 2, wherein the covering portion includes an elastic portion configured to be expanded and contracted between an expanded state where it is expanded to cover a surface of the medical equipment and a contracted state where the medical equipment surface covering state is released.

4. The drape for the medical equipment according to claim 1, wherein the covering portion includes an elastic portion configured to be expanded and contracted between an expanded state where it is expanded to cover a surface of the medical equipment and a contracted state where the medical equipment surface covering state is released.

5. A drape for an endoscope comprising:
a covering portion configured to change from a state where at least a part of an operation portion of the endoscope having an insertion portion and the operation portion provided at a proximal end portion of the insertion portion is covered to a state where the entire insertion portion is covered;
a fixing portion which relatively fixes the covering portion to the endoscope;
a coupling portion which couples the fixing portion to the covering portion; and
holding means which is provided to the covering portion and configured to liquid-tightly cover the entire insertion portion in a state where the entire insertion portion is covered with the covering portion.

6. A medical apparatus comprising:
a control apparatus:
an endoscope having an insertion portion and an operation portion which is provided at a proximal end portion of the insertion portion and has an attachment/detachment portion detachably connected with the control apparatus;
a covering portion configured to change from a state where at least a part of the operation portion is covered to a state where the entire insertion portion is liquid-tightly covered;
a fixing portion which relatively fixes the covering portion to the attachment/detachment portion; and
holding means for enabling the covering portion to cover a surface of the control apparatus.
